(19) United States
(12) Patent Application Publication
(10) Pub. No.: US 2026/0201142 A1
(43) Pub. Date: Jul. 16, 2026

(54) METHOD FOR REMOVING POLYMERS INSIDE OLEFIN OLIGOMERIZATION REACTOR

(71) Applicant: HANWHA TOTALENERGIES PETROCHEMICAL CO., LTD., Seosan-si Chungcheongnam-do (KR)

(72) Inventors: Wonjong HONG, Seosan-si Chungcheongnam-do (KR); Kyesung CHO, Seosan-si Chungcheongnam-do (KR); Young Hoon SOHN, Seosan-si Chungcheongnam-do (KR); Minsu KO, Seosan-si Chungcheongnam-do (KR); Jinsuk LEE, Seoul (KR); Sangjoon OH, Seosan-si Chungcheongnam-do (KR); Hosik CHANG, Daejeon (KR)

(21) Appl. No.: 19/132,403

(22) PCT Filed: Sep. 12, 2023
(86) PCT No.: PCT/KR2023/013611

§ 371 Date: May 23, 2025

(30) Foreign Application Priority Data
Dec. 26, 2022 (KR) 10-2022-0184717

(51) Int. Cl. C08J 11/08; C07C 7/04

(52) U.S. Cl. CPC C08J 11/08; C07C 7/04

(57) ABSTRACT

The present invention relates to a method for removing polymers which are generated during olefin oligomerization and which are accumulated inside a reactor and cause fouling, the method enabling simultaneous oligomerization and polymer removal so that process operation can be efficient, requiring no separate heat source or cooling water such that economic feasibility is improved, and rapidly cooling a polymer solution such that polymers are readily removed from a solvent, thereby enabling solvent recovery rate to be improved.

### TECHNICAL FIELD

The present invention relates to a method for removing polymers in an olefin oligomerization reactor, and more particularly, to a method for removing polymers in an olefin oligomerization reactor, which may effectively remove polymers which accumulate inside the reactor and cause fouling, without process interruption.

### BACKGROUND ART

In an oligomerization reaction process of an olefin, a large amount of polymers such as polyethylene may be produced as by-products, in addition to an oligomer such as 1-hexene and 1-octene, which are a target material. The polymers are attached to piping and reactors to block piping, or the polymers accumulated in the reactor may reduce a flow velocity of a fluid, serve as an insulation, and interfere with heat transfer. In order to remove polymers which adversely affect the process, conventionally, the reactor is opened after process shut down and the polymers inside the reactor are directly removed by a worker. However, since this requires a large amount of manpower, increases working time, and needs an additional process such as removing impurities from a reactor for process restart and then performing substitution with inert gas, process efficiency and economic feasibility are poor.

Therefore, there is a need to study a method for removing polymers in an oligomerization reactor, in which a reactor is not opened, a process of removing polymers in the reactor simultaneously with performing an olefin oligomerization reaction is continuously performed to have higher solvent recovery rate, and thus, efficient and economical operation is allowed.

### DISCLOSURE

### Technical Problem

In order to solve the problems of the conventional art, an object of the present invention is to provide a method for removing polymers in an oligomerization reactor, which may effectively remove polymers, which are by-products of an olefin oligomerization reaction and accumulate in the reactor and cause fouling, without process interruption.

Another object of the present invention is to provide a method for removing polymers in an oligomerization reactor and an oligomer manufacturing device, which may improve a solvent recovery rate in a polymer removal process.

### Technical Solution

In one general aspect, a method for removing polymers in an oligomerization reactor includes: (a) preparing an ethylene-based oligomer from reactants including ethylene, a solvent, and a catalyst in a reactor; (b) removing polymers included in an oligomer product solution; (c) distilling the oligomer product solution from which the polymers have been removed in (b) in a distillation tower to separate the solution into C4 or lower hydrocarbons, 1-hexene, 1-octene, C10 or higher hydrocarbons, and the solvent; (d) recovering a heated recovered solvent obtained by heating a part of the separated solvent of (c) to the reactor to dissolve residual polymers in the reactor to prepare a polymer dissolved solution; (e) mixing the polymer dissolved solution and the remaining recovered solvent of (c), and rapidly cooling the polymer dissolved solution to precipitate the polymers; and (f) separating the polymers from the solvent, in the polymer dissolved solution from which the polymers have been precipitated.

In an exemplary embodiment, in step (d), the part of the separated solvent may be heated by heat exchange with the C10 or higher hydrocarbons separated from the distillation tower using a heat exchanger.

In an exemplary embodiment, in step (d), a temperature of the heated recovered solvent may be 50 to 200° C.

In an exemplary embodiment, in step (d), a temperature of the C10 or higher hydrocarbons may be 150 to 300° C.

In an exemplary embodiment, in step (e), a temperature of the remaining recovered solvent may be 30 to 70° C.

In an exemplary embodiment, in step (e), a temperature of the rapidly cooled polymer dissolved solution may be 20 to 80° C.

In an exemplary embodiment, in step (e), a mixing ratio between the polymer dissolved solution and the remaining recovered solvent may be 1:1 to 7.

In an exemplary embodiment, in step (d), a flow rate ratio between the part of the separated solvent and the C10 or higher hydrocarbons may be 1:0.5 to 3.

In an exemplary embodiment, a flow rate ratio between the heated recovered solvent and the remaining recovered solvent of the separated solvent of step (c) may be 1:20 to 150.

In an exemplary embodiment, in step (e), the polymer dissolved solution and the remaining recovered solvent may be mixed in a polymer precipitation device.

In an exemplary embodiment, in step (f), the solvent separated from the polymer separator may be recovered and reused in the oligomerization reaction of step (a).

In an exemplary embodiment, after step (b), a monomer recovery step of separating unreacted monomers included in the oligomer from which the polymers have been removed using a gas-liquid separator and recovering the monomers to the reactor may be further included.

In an exemplary embodiment, the solvent may include an aliphatic hydrocarbon or an aromatic hydrocarbon.

In an exemplary embodiment, in step (a), a plurality of reactors may be included.

In an exemplary embodiment, the plurality of reactors may be arranged in parallel.

In an exemplary embodiment, after step (d), a step of transferring the part of the heated recovered solvent to the polymer removal device and dissolving a residual polymer in the polymer removal device to prepare a polymer dissolved solution may be further included.

In an exemplary embodiment, after step (a), a step of injecting a catalyst deactivator into the oligomer product solution may be further included.

In an exemplary embodiment, the catalyst deactivator may include water, an alcohol-based compound, or a combination thereof.

### Advantageous Effects

Since the method for removing polymers in an olefin oligomerization reactor according to the present invention may remove polymers accumulated in the reactor continuously while simultaneously performing an oligomerization reaction, it may improve process efficiency.

In addition, a solvent used in the oligomerization reaction may be recovered and used for removing polymers accumulated in the reactor, and during polymer removal, heating may be performed by heat exchange with hydrocarbons having a high-boiling point at a high temperature which is produced in the process without a need to add a separate heat source. Thus, excellent economic feasibility may be provided.

Furthermore, when the polymers are precipitated, rapid cooling is performed, so that the polymers and the solvent may be effectively separated, and thus, a polymer removal rate and a solvent recovery rate may be improved.

### DESCRIPTION OF DRAWINGS

FIG. 1 is a conventional oligomerization reaction process diagram of an olefin.

FIG. 2 is an olefin oligomerization reaction process diagram according to Example 1.

FIG. 3 is a drawing showing flows of an oligomerization reaction process (blue dotted line) and a polymer removal process (red solid line), in the oligomerization reaction process diagram according to Example 1.

FIG. 4 is a drawing showing a difference in agglomeration of a polymer depending on a cooling rate of a polymer dissolved solution.

FIG. 5 is a process diagram showing an embodiment in which the number of reactors is 3, in the olefin oligomerization reaction process according to Example 1.

FIG. 6 is a process diagram showing an embodiment including a process of removing polymers accumulated in a polymer removal device, in the olefin oligomerization reaction process according to Example 1.

### BEST MODE

A method for removing polymers in an olefin oligomerization reactor of the present invention will be described in detail. The terms used in the present specification are selected to be as common as possible and are currently widely used while considering the function of the present invention, but they may vary depending on the intention of a person skilled in the art, a convention, the emergence of new technology, or the like. The technical and scientific terms used may have, unless otherwise defined, the meaning commonly understood by those of ordinary skill in the art.

The terms such as “comprise” or ““have” in the present specification and the appended claims mean that there is a characteristic or a constitutional element described in the specification, and as long as it is not particularly limited, a possibility of adding one or more other characteristics or constitutional elements is not excluded in advance.

In the present specification and the appended claims, the terms such as “first” and “second” are not used in a limited meaning but are used for the purpose of distinguishing one constituent element from other constituent elements.

A singular expression in the present specification and the appended claims includes a plural expression, unless otherwise explicitly specified as singular. In addition, a plural expression includes a singular expression, unless otherwise explicitly specified as plural.

In the present specification and the appended claims, the terms such as “first” and “second” are not used in a limited meaning but are used for the purpose of distinguishing one constituent element from other constituent elements.

In addition, the numerical range used in the present specification includes all values within the range including the lower limit and the upper limit, increments logically derived in a form and span of a defined range, all double limited values, and all possible combinations of the upper limit and the lower limit in the numerical range defined in different forms. Unless otherwise defined in the specification of the present invention, values which may be outside a numerical range due to experimental error or rounding off of a value are also included in the defined numerical range.

The terms of degree “about” and the like used in the present specification and the attached claims are used in the sense of covering an allowable error when the allowable error exists.

Hereinafter, the present invention will be described in more detail with reference to the attached drawings.

In order to prepare an ethylene-based oligomer, ethylene, a solvent, and a catalyst are added to a reactor to perform an oligomerization reaction. Polymers such as polyethylene may be produced as a by-product, in addition to α-olefins such as 1-hexene and 1-octene which are a target material, by trimerization and tetramerization reactions of ethylene. Polyethylene, which is a by-product, accumulates in the inner wall or piping of the reactor as the reaction continues to be a cause of pipe blockage, lowers the velocity of a fluid, and may act as an insulation which interferes with heat transfer of the reactor. Therefore, it is important to remove polymers accumulated in the inner wall of the reactor.

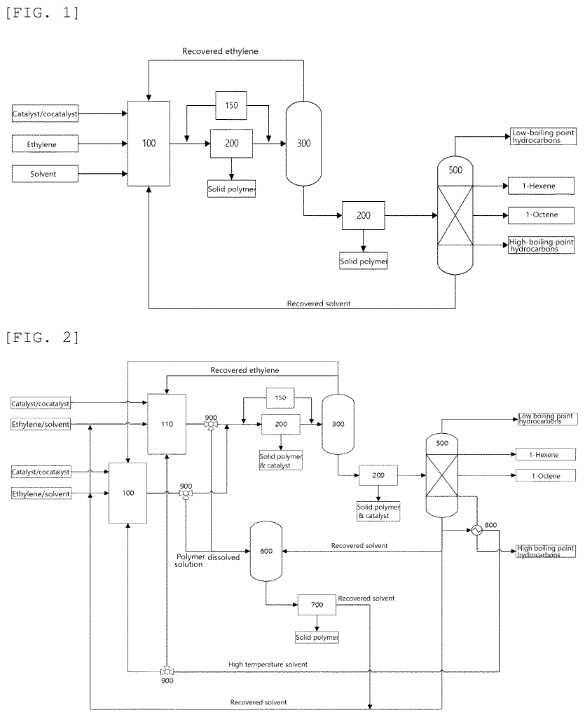

However, as shown in FIG. 1, in a conventional common oligomerization reaction process of ethylene, since there is no separate process of removing polymers formed in the inner wall of the reactor, the process is stopped and the reactor is opened to remove polymers directly by a worker inside the reactor. The method requires a large amount of manpower, increases working time, and needs an additional process such as removing impurities from a reactor for process restart and then performing substitution with inert gas, process efficiency and economic feasibility are very poor.

Thus, after an in-depth study, the present applicant removed residual polymers in the inner wall of the reactor by recovering a solvent used in the reaction to a reactor without opening the reactor to dissolve polymers accumulated in the inner wall of the reactor, and effectively separated the polymers from the solvent by rapidly cooling the solvent dissolving the polymer, thereby inventing a method for removing polymers in the olefin oligomerization reactor with an improved solvent recovery rate.

Hereinafter, the present invention will be described in more detail with reference to the attached drawings.

As shown in FIG. 2, the method for removing polymers in an oligomerization reactor according to the present invention includes: (a) preparing an ethylene-based oligomer from reactants including ethylene, a solvent, and a catalyst in a reactor; (b) removing polymers included in an oligomer product solution; (c) distilling the oligomer product solution from which the polymers have been removed in (b) in a distillation tower to separate the solution into C4 or lower hydrocarbons, 1-hexene, 1-octene, C10 or higher hydrocarbons, and the solvent; (d) recovering a heated recovered solvent obtained by heating a part of the separated solvent of (c) to the reactor to dissolve residual polymers in the reactor to prepare a polymer dissolved solution; (e) mixing the polymer dissolved solution and the remaining recovered solvent of (c), and rapidly cooling the polymer dissolved solution to precipitate the polymers; and (f) separating the polymers from the solvent, in the polymer dissolved solution from which the polymers have been precipitated.

In step (a), reactants including ethylene, a solvent, and a catalyst may be added to a reactor 110 to form an ethylene-based oligomer through an oligomerization reaction.

The solvent may include an aliphatic hydrocarbon or an aromatic hydrocarbon. Specifically, the aliphatic hydrocarbon solvent may be an aliphatic hydrocarbon solvent having 3 to 20 carbon atoms or an aromatic hydrocarbon solvent having 6 to 20 carbon atoms.

More specifically, the hydrocarbon solvent may be selected from the group consisting of toluene, xylene, chlorobenzene, dichlorobenzene, dichloromethane, hexane, methylcyclohexane, and cyclohexane, and preferably, the solvent may include methylcyclohexane. When the solvent is used, polymerization activity may be high, it is easy to separate the product and the solvent after the olefin oligomerization reaction, and it is easy to dissolve polymers.

The catalyst may be a catalyst used in a common olefin oligomerization process, but preferably a chromium-based catalyst may be included. The reactants may further include a cocatalyst. For example, the cocatalyst may include an organic aluminum compound, but the present invention is not limited thereto. By including the catalyst, the ethylene-based oligomer may be prepared with high selectivity and conversion rate with excellent catalytic activity even at a low temperature.

In step (a), the reactants are added to the reactor 110 to perform trimerization and tetramerization reactions of ethylene may be performed. Herein, before injecting ethylene and a solvent into the reactor 110, a pretreatment process may be performed. The pretreatment process may be commonly performed by an adsorption process, and oxygen and moisture contained in ethylene and the solvent may be removed by a pretreatment process.

A plurality of reactors may be included, and the plurality of reactors may be arranged in parallel. In a part of the plurality of reactors arranged in parallel, an oligomerization reaction process is performed, and simultaneously, in reactors which do not perform the oligomerization reaction process, polymers accumulated in the inner wall of the reactor are removed to clean the reactor.

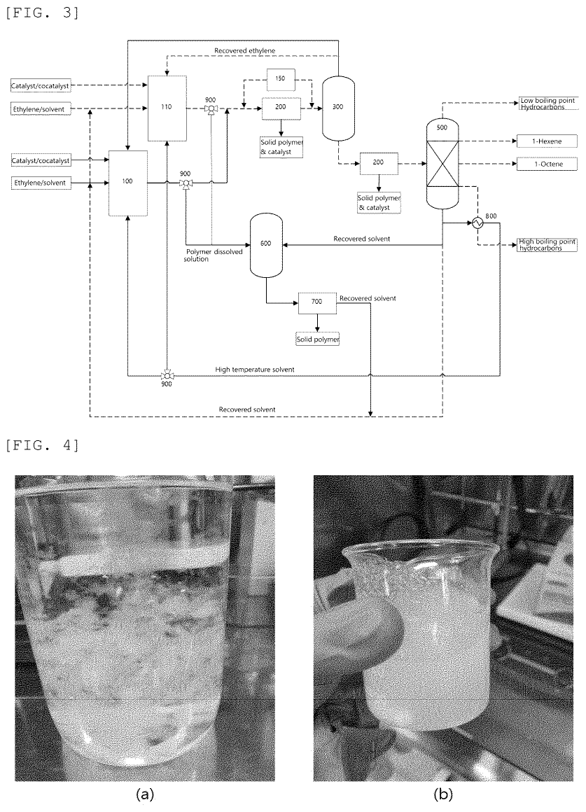

More specifically, referring to FIG. 3, a second reactor 110 performs the oligomerization reaction process through a plurality of reactors, and during preparation of the ethylene-based oligomer, a process of removing polymers in which polyethylene accumulated in the inner wall of a first reactor 100 is removed may be performed. On the contrary, while the process of preparing an ethylene-based oligomer proceeds in the first reactor 100, the process of removing polyethylene in the second reactor 110 may be performed.

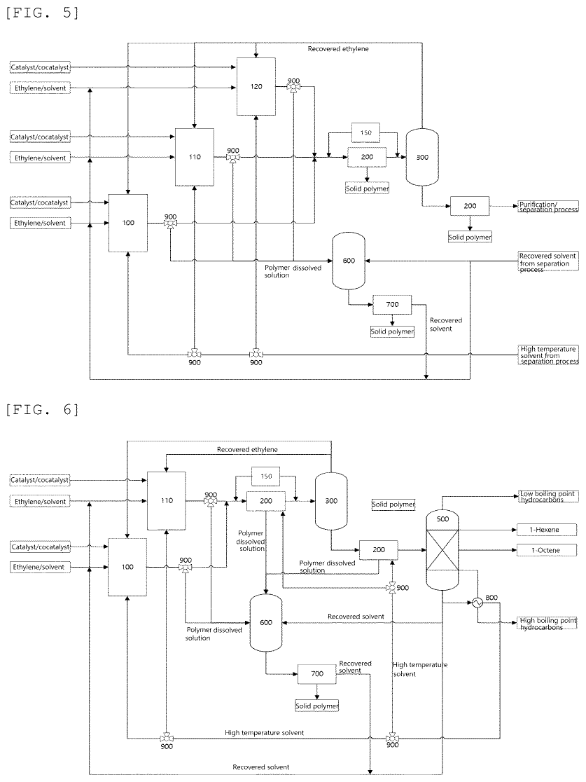

Otherwise, as shown in FIG. 5, three reactors may be arranged in parallel. In the second reactor 110 and a third reactor 120, the ethylene oligomerization process is performed, and simultaneously, in the first reactor 100, a process of removing polymers accumulated in the inner wall of the reactor is performed, thereby continuously preparing α-olefin. Therefore, since the process of preparing an oligomer and the process of removing polymers may be performed continuously and simultaneously without stopping the process, process efficiency may be improved.

The reactor may be one or more reactors selected from the group consisting of a batch reactor, a continuous stirred tank reactor, a tubular reactor, a loop reactor, a bubble top reactor, and a fluidized bed reactor, and preferably, a continuous stirred tank reactor may be used.

An oligomer product solution prepared from step (a) may include α-olefins including 1-hexene and 1-octene, unreacted ethylene, polyethylene, and methylcyclohexane. In addition to the α-olefin which is a target material, polymers including polyethylene are produced and accumulate in the inner wall and piping of the reactor, resulting in process interruption. Thus, since the solvent used in an oligomerization process is recovered and the polymers inside the reactor are rapidly removed, as described later, fouling may be effectively suppressed without process interruption.

After step (a), a step of deactivating the catalyst by injecting a catalyst deactivator into the oligomer product solution with a catalyst deactivator injector 150 may be further included. The catalyst deactivator injector 150 may be placed at the front or rear end of a polymer removal device 200. After deactivating a residual catalyst included in the oligomer product solution through the catalyst deactivator, the residual catalyst and the catalyst deactivator may be discharged from the polymer removal device 200 to improve the recovery efficiency of the solvent.

The catalyst deactivator may be any material known in the art. Specifically, the catalyst deactivator may include water, an alcohol-based compound, or a combination thereof, preferably may include 2-ethylhexanol as the alcohol-based compound.

Polyethylene included in the oligomer product solution described above may be removed with a polymer removal device 200 in step (b). The polymer removal device 200 may remove polyethylene using one selected from the group consisting of centrifugal separation, compression filtration, gravity filtration, a metal filter, a ceramic membrane filter, a sand filter, and an adsorption device, but the present invention is not limited to a specific type of polymer removal device 200.

In an exemplary embodiment, after step (b), a step of removing unreacted ethylene included in the oligomer product solution from which the polymers have been removed with a gas-liquid separator 300 may be further included. The unreacted ethylene separated in the gas-liquid separator 300 is recovered to the reactor and reused in the ethylene oligomerization reaction of step (a). As a non-limiting example, a plurality of gas-liquid separators 300 may be installed continuously.

After the step of removing unreacted ethylene, a polymer removal device 200 may be further arranged to further perform step (b). The plurality of polymer removal devices 200 may be arranged to effectively remove polyethylene included in the oligomer product solution.

In an exemplary embodiment, in step (c), an oligomer product solution from which polyethylene and unreacted ethylene have been removed may be distilled in a distillation tower of a distillation device 500 to separate the solution into C4 or lower hydrocarbons, 1-hexene, 1-octene, C10 or higher hydrocarbons, and a solvent. The C4 or lower hydrocarbons may include hydrogen, ethylene, and 1-butene, and the distillation device 500 may include a plurality of distillation towers and separate the oligomer product solution depending on the boiling point, respectively.

In an exemplary embodiment, in step (d), a part of the solvent separated in the distillation device 500 through step (c) is heated to form a heated recovered solvent which is recovered to the reactor, and residual polyethylene accumulated in the inner wall of the reactor 100 may be dissolved with the heated recovered solvent to prepare a polymer dissolved solution.

Herein, it is preferred that heating of the solvent is performed by heat exchange with C10 or higher hydrocarbons at a high temperature separated in the distillation device 500 using a heat exchanger 800. Conventionally, low pressure steam is mainly used for heating a solvent, and when a part of the solvent separated using low pressure stem is heated to a high temperature, a large amount of fluid is required. However, when the solvent is heated using the heat exchanger 800 as in the present invention, the solvent may be heated to a high temperature only with a significantly smaller amount of the fluid as compared with low pressure steam, which is advantageous.

In an exemplary embodiment, in step (d), a flow rate ratio between the part of the separated solvent and the C10 or higher hydrocarbons may be 1:0.5 to 3, 1:0.7 to 3, or 1:1 to 3, and preferably 1:1 to 2. At the flow rate ratio, the solvent may be heated to a temperature at which the solvent may dissolve the polymer.

More specifically, the temperature of the C10 or higher hydrocarbons may be 150 to 300° C., 160 to 270° C., 170 to 240° C., or 180 to 220° C., and substantially, may be 190 to 200° C. The temperature of the heated recovered solvent which is heated by heat exchange with the C10 or higher hydrocarbons having the temperature range described above may be 50° C. to 200° C., 55° C. to 180° C., or 60° C. to 150° C., and substantially, the solvent may be heated to 70° C. to 110° C. to easily dissolve polyethylene accumulated in the inner wall of the reactor.

Through the heat exchange with the C10 or higher hydrocarbons, the solvent may be simply heated without adding a separate heating process and equipment to effectively save energy used in the oligomerization reaction process.

In an exemplary embodiment, the C10 or higher hydrocarbons may be cooled to 250° C. or lower, 230° C. or lower, 200° C. or lower, 180° C. or lower, 150° C. or lower, or 130° C. or lower, and advantageously 100° C. or lower by heat exchange with the solvent. Cooling water usage may be significantly reduced by the process of heat exchange with the solvent using the heat exchanger 800. In general, in order to store the C10 or higher hydrocarbons at a high temperature, an additional cooling process is needed. However, when heat exchange with the solvent is performed using the heat exchanger 800, the C10 or higher hydrocarbons are cooled to about 100° C. or lower to reduce the cooling water usage, which is advantageous in terms of energy efficiency.

In an exemplary embodiment, in step (e), the polymer dissolved solution obtained by dissolving residual polyethylene accumulated in the inner wall of the reactor 100 and the remaining recovered solvent separated in the distillation device 500 may be mixed, and the polymer dissolved solution may be rapidly cooled to precipitate polyethylene.

In the step of rapidly cooling the polymer dissolved solution, an additional cooling source may not be needed. Since the recovered solvent remaining after being used in the oligomer preparation reaction may be used without a need to provide a separate cooling solution to perform rapid cooling, excellent energy efficiency and economic feasibility may be provided.

In an exemplary embodiment, the temperature of the remaining recovered solvent may be 30 to 70° C., 35 to 65° C., 40 to 60° C., 45 to 55° C., or 45 to 50° C. When the recovered solvent having a lower temperature than the polymer dissolved solution is mixed with the polymer dissolved solution, the polymer dissolved solution is rapidly cooled so that the polymer in the polymer dissolved solution may rapidly agglomerate. The agglomerated polymer may be precipitated, and polyethylene may be separated from the solvent and easily removed.

The polymer dissolved solution and the remaining recovered solvent may be mixed at a mixing ratio of 1:1 to 7, 1:1 to 6, or 1:2 to 5, preferably at a mixing ratio of 1:2 to 4. By mixing the polymer dissolved solution and the remaining recovered solvent at the ratio, the polymer dissolved solution may be rapidly cooled to secure a cooling temperature at which polyethylene may be easily precipitated.

Specifically, the temperature of the polymer dissolved solution after rapid cooling may be 20 to 80° C., 30 to 77° C., 40 to 75° C., 50 to 75° C., or 60 to 70° C.

When the polymer dissolved solution at a high temperature is naturally cooled, the polymer does not agglomerate well and dispersed in the solvent in the form such as gel, so that it may be difficult to separate the polymer from the solvent and remove it. However, when the polymer dissolved solution is rapidly cooled with the remaining recovered solvent at a low temperature, the polymer may easily agglomerate into a solid phase. Since the size of the polymer particles agglomerated in the rapidly cooled polymer dissolved solution is larger than that in the naturally cooled polymer dissolved solution, the polymer may be easily separated from the solvent and removed.

In an exemplary embodiment, a flow rate ratio of the heated recovered solvent: the remaining recovered solvent of the solvent separated in step (c) may be 1:20 to 150, 1:30 to 140, 1:40 to 130, or 1:50 to 120. The solvent is separated at the flow rate ratio, the residual polymer in the inner wall of the reactor 100 is removed with the heated recovered solvent to prepare the polymer dissolved solution, and the polymer dissolved solution is rapidly cooled with the remaining recovered solvent to easily precipitate the polymer.

In another exemplary embodiment, in step (d), a step of transferring the part of the heated recovered solvent to the polymer removal device to prepare a polymer dissolved solution in which the residual polymer is dissolved in the polymer removal device may be further included.

Specifically, referring to FIG. 6, a first transfer line through which a part of the heated recovered solvent is transferred to the polymer removal device 200, and a second transfer line through which the polymer dissolved solution produced in the polymer removal device 200 is transferred to the polymer precipitation device 600 may be further included.

A part of the heated recovered solvent recovered to the reactor 100 may be transferred to the polymer removal device 200 to dissolve the polymers accumulated in the inner wall of the polymer removal device 200. The produced polymer dissolved solution may move from the polymer removal device 200 to the polymer precipitation device 600 through the second transfer line. The polymer dissolved solution transferred to the polymer precipitation device 600 may be precipitated through rapid cooling, and the polymer may be separated from the polymer dissolved solution and removed.

When the olefin oligomerization process is performed by the method for removing polymers in the oligomerization reactor of the present invention, the polymer accumulated in a plurality of polymer removal devices 200 as well as the first reactor 100 and the second reactor 110 may also be easily removed with a heated recovered solvent, which is advantageous.

In an exemplary embodiment, in step (f), in the polymer dissolved solution from which the polymer has been precipitated by rapid cooling in step (e), the polymer may be separated from the solvent through a polymer separator 700.

The solvent separated through the polymer separator 700 may be recovered to step (a) and reused in preparing an ethylene-based oligomer. When the solvent obtained after passing through the polymer separator 700 is recovered and reused in the oligomerization reaction, a separate pretreatment process for removing oxygen and moisture included in the solvent is not needed, which is more efficient.

As a non-limiting example, a plurality of polymer separators 700 may be arranged in parallel, so that the polymer removal effect may be further improved.

In an exemplary embodiment, the polymer separators 700 may include one selected from a centrifuge, a 3-phase separator, a filter press, a screw press, a screw decanter, a screen, a drum flaker, and a filter, but the present invention is not limited thereto.

Hereinafter, the present invention will be described in detail by the examples and the comparative examples.

### Preparation Example 1

An oligomerization reaction process and a polymer removal process in a reactor were performed according to the process diagram shown in FIG. 2.

### Ethylene-Based Oligomer Preparation Step

1.5 L/hr of methylcyclohexane, 250 g/hr of ethylene, 0.02 μmol/hr of a catalyst, and 7 μmol/hr of a cocatalyst were injected into a first reactor 100 and a second reactor 110 which were 2 L continuous stirred tank reactors heated to 40° C. In a state in which the reaction temperature was maintained at 40° C. while ethylene was continuously supplied so that the internal pressure of the reactor was maintained at 30 bar, the reaction was performed for 90 hours, the injection of the reactants into the first reactor 100 was blocked, the product inside the reactor was discharged, and the reaction was ended.

### Removal and Precipitation Step of Residual Polymer in Reactor

After the oligomer preparation step, the product was separated into C4 or lower low-boiling point hydrocarbons, 1-hexene, 1-octene, C10 or higher high-boiling point hydrocarbons, and methylcyclohexane. A heated recovered solvent which was heated to 110° C. by heat exchange of a part of the separated methylcyclohexane with high-boiling point hydrocarbons was injected into the first reactor, and stirring was performed for 20 minutes to prepare a polymer dissolved solution in which residual polyethylene accumulated inside the reactor was dissolved.

Thereafter, the polymer dissolved solution was mixed with the remaining recovery methylcyclohexane to rapidly cool the polymer dissolved solution. The rapid cooling was performed for about 10 minutes, and polyethylene was precipitated from the cooled polymer dissolved solution. In the polymer dissolved solution including the precipitated polyethylene, polyethylene was separated from methylcyclohexane through a centrifuge.

The composition of the product obtained in the oligomerization reaction process was analyzed and heat and material balance in the polymer removal device 200, a gas-liquid separator 300, a distillation device 500, and the heat exchanger 800 was derived using a chemical process simulation software (Aspen Plus v.11).

The temperature of the remaining recovery methylcyclohexane derived through the process simulation software was 52° C., and the temperature of C10 or higher hydrocarbons discharged in the lower portion of the high boiling point separation distillation tower was 196° C.

### Example 1

When the ethylene oligomerization process was performed by the method of Preparation Example 1, a part of the separated methylcyclohexane was heat exchanged with C10 or higher high-boiling point hydrocarbons at a flow rate ratio of 1:2 to prepare a heated recovered solvent. In addition, during rapid cooling of the polymer dissolved solution in the polymer precipitation device 600, the polymer dissolved solution and the remaining recovery methylcyclohexane were mixed at a mixing ratio of 1:2 to perform the ethylene oligomerization process.

### Example 2

The ethylene oligomerization process was performed in the same manner as in Example 1, except that during the rapid cooling in the polymer precipitation device 600, the polymer dissolved solution and the remaining recovery methylcyclohexane were mixed at a ratio of 1:1.

### Example 3

The ethylene oligomerization process was performed in the same manner as in Example 1, except that during the rapid cooling in the polymer precipitation device 600, the polymer dissolved solution and the remaining recovery methylcyclohexane were mixed at a ratio of 1:3.

### Example 4

The oligomerization process of ethylene was performed in the same manner as in Example 1, except that during the rapid cooling in the polymer precipitation device 600, the polymer dissolved solution and the remaining recovery methylcyclohexane were mixed at a ratio of 1:4.

### Example 5

The ethylene oligomerization process was performed in the same manner as in Example 1, except that during the rapid cooling in the polymer precipitation device 600, the polymer dissolved solution and the remaining recovery methylcyclohexane were mixed at a ratio of 1:5.

### Comparative Example 1

The ethylene oligomerization process was performed in the same manner as in Example 1, except that the polymer dissolved solution was naturally cooled from room temperature to 25° C. in the polymer precipitation device 600.

### Comparative Example 2

The ethylene oligomerization process was performed in the same manner as in Example 1, except that a part of the separated methylcyclohexane was heated with low pressure steam to prepare the heated recovered solvent. The pressure of the low pressure steam was 3.3 barg and the temperature was 147° C.

### Experimental Example 1

### Evaluation of Agglomeration of Polymer Depending on Cooling Rate of Polymer Dissolved Solution

The ethylene oligomerization process was performed according to Example 1 and Comparative Example 1, the agglomeration of the cooled polymer dissolved solution was observed and shown in FIG. 4, and the amount of the polymer separated in the polymer separator 700 is shown in the following Table 1.

TABLE 1

| | Amount (g) of precipitated polymer |
|---|---|
| Example 1 | 7.8 |
| Comparative Example 1 | 3.2 |

As shown in FIG. 4, it was confirmed that in the polymer dissolved solution (a) which was rapidly cooled according to Example 1, the polymers were agglomerated and separated into polymers and a solvent, but in the polymer dissolved solution (b) which was naturally cooled according to Comparative Example 1, the solvent and the polymers were not separated and the polymer were dispersed in a gel form in the solvent. When the polymer dissolved solution was naturally cooled as in Comparative Example 1, the size of the agglomerated polymer particles was very small and dispersed in the solvent, so that it was difficult to separate the polymers from the solvent. Therefore, the amount of polymers precipitated in the polymer separator 700 was very small as compared with Example 1.

However, in the polymer dissolved solution which as rapidly cooled in Example 1, the polymers were agglomerated and separated from the solvent, and the size of the agglomerated polymer particles was large so that the solvent and the polymer were easily separated in the polymer separator 700 and it was easy to remove the polymers. As the amount of polymers separated in the polymer separator 700 was increased, the amount of the solvent recovered to the reactor was also increased. The solvent recovered to the reactor was able to be reused in the ethylene oligomerization reaction, which was advantageous.

### Experimental Example 2

### Evaluation of Cooling Temperature Depending on Mixing Ratio of Heated Recovered Solvent and Polymer Dissolved Solution

In the ethylene oligomerization process according to Examples 1 to 4, during rapid cooling of the polymer dissolved solution, the temperature after mixing according to the mixing ratio between the remaining recovered solvent and the polymer solution was derived through a chemical process simulation software (Aspen Plus v.11) and is shown in the following Table 2.

TABLE 2

| | Polymer dissolved | |
|---|---|---|
| | solution:remaining | Temperature (° C.) |
| | recovered solvent | after mixing |
| Example 1 | 1:2 | 70 |
| Example 2 | 1:1 | 81 |
| Example 3 | 1:3 | 66 |
| Example 4 | 1:4 | 62 |
| Example 5 | 1:5 | 60 |

In all of Examples 1 to 5, when the remaining recovered solvent was mixed with the polymer dissolved solutions at 110° C., it was confirmed that the polymer dissolved solution was rapidly cooled, so that the temperature at which the polymers in the polymer dissolved solution may be precipitated was created.

As shown in (a) of FIG. 4, in Example 1, the polymer dissolved solution was rapidly cooled to 70° C., and the size of the particles of the agglomerated polymer was increased. Accordingly, the precipitated polymers were easily separated in the polymer separator 700.

When the polymer dissolved solution was rapidly cooled, as the amount of the remaining recovered solvent to be mixed increased, the temperature of the polymer dissolved solution after mixing was lowered. In Examples 1 and 3 to 5 in which the amount of the remaining recovered solvent to be mixed was twice the amount of the polymer dissolved solution, the polymer dissolved solution was rapidly cooled, so that the temperature at which the polymers may be effectively agglomerated was able to be secured.

In addition, in Example 1, though a smaller amount of the remaining recovered solvent than Examples 3 to 5 was mixed with the polymer dissolved solution, the polymer dissolved solution was rapidly cooled to form a temperature at which the polymer may be easily precipitated. Accordingly, Example 1 in which the mixing ratio between the polymer dissolved solution and the remaining recovered solvent was 1:2 saved the remaining recovered solvent usage to provide excellent economic feasibility.

### Experimental Example 3

### Evaluation of Heating Fluid Amount Required Depending on Fluid Heating Part of Solvent

When the ethylene oligomerization process was performed according to the methods according to Example 1 and Comparative Example 2, the amount of heating fluid required depending on the method for heating a part of methylcyclohexane separated in the distillation device 500 was derived through the chemical process simulation software (Aspen Plus v.11) and is shown in the following Table 3.

TABLE 3

| | Solvent heating | Amount of heating fluid |
|---|---|---|
| | method | required per unit solvent |
| Example 1 | Heat exchange | 2 |
| | with C10 or higher | |
| | hydrocarbons | |
| Comparative | Low pressure | 10 |
| Example 2 | steam | |

When a part of methylcyclohexane separated in the distillation device was heated at the same temperature (110° C.), it was confirmed that the amount of the heated fluid required in Example 1 was significantly decreased as compared with Comparative Example 2. As a result of process simulation, since the temperature of C10 or higher hydrocarbons (196° C.) of Example 1 was higher than the low pressure steam (147° C.), the solvent was able to be heated to a high temperature only with a small amount of fluid.

In order to heat the solvent with low pressure steam as in Comparative Example 2, an additional facility was needed, and also a large amount of fluid and energy were consumed. However, when the oligomerization reaction process was performed by the method of Example 1, the solvent was heated to a target temperature with a small amount of fluid and energy, through a process of heat exchange with C10 or higher hydrocarbons produced in the process using a heat exchanger.

Furthermore, the C10 or higher hydrocarbons at a high temperature were also cooled from 196° C. to about 100° C. only using a heat exchange process with the solvent without a need to cool using separate cooling water. Therefore, the amount of fluid, cooling water usage, and consumed energy required for heating the solvent were able to be saved, which is advantageous.

Hereinabove, although the present invention has been described by specific matters, limited exemplary embodiments, and drawings, they have been provided only for assisting the entire understanding of the present disclosure, and the present disclosure is not limited to the exemplary embodiments, and various modifications and changes may be made by those skilled in the art to which the present disclosure pertains from the description.

Therefore, the spirit of the present invention should not be limited to the above-described exemplary embodiments, and the following claims as well as all modifications equal or equivalent to the claims are intended to fall within the scope and spirit of the invention.

### DETAILED DESCRIPTION OF MAIN ELEMENTS

100: First reactor 110: Second reactor
120: Third reactor 150: Catalyst deactivator injector
200: Polymer removal device 300: Gas-liquid separator
500: Distillation device 600: Polymer precipitation device
700: Polymer separator 800: Heat exchanger
900: Valve

1. A method for removing polymers in an oligomerization reactor, the method comprising:
(a) preparing an ethylene-based oligomer from reactants including ethylene, a solvent, and a catalyst in a reactor;
(b) removing polymers included in an oligomer product solution;
(c) distilling the oligomer product solution from which the polymers have been removed in (b) to separate the solution into C4 or lower hydrocarbons, 1-hexene, 1-octene, C10 or higher hydrocarbons, and the solvent;
(d) recovering a heated recovered solvent obtained by heating a part of the separated solvent of (c) to the reactor to dissolve residual polymers in the reactor to prepare a polymer dissolved solution;
(e) mixing the polymer dissolved solution and the remaining recovered solvent of (c), and rapidly cooling the polymer dissolved solution to precipitate the polymers; and
(f) separating the polymers from the solvent, in the polymer dissolved solution from which the polymers have been precipitated.

2. The method for removing polymers in an oligomerization reactor of claim 1, wherein in (d), the part of the separated solvent is heated by heat exchange with the C10 or higher hydrocarbons separated from the distillation tower using a heat exchanger.

3. The method for removing polymers in an oligomerization reactor of claim 1, wherein in (d), a temperature of the heated recovered solvent is 50° C. to 200° C.

4. The method for removing polymers in an oligomerization reactor of claim 2, wherein in (d), a temperature of the C10 or higher hydrocarbons is 150 to 300° C.

5. The method for removing polymers in an oligomerization reactor of claim 4, wherein in (e), a temperature of the remaining recovered solvent is 30 to 70° C.

6. The method for removing polymers in an oligomerization reactor of claim 4, wherein in (e), a temperature of the rapidly cooled polymer dissolved solution is 20 to 80° C.

7. The method for removing polymers in an oligomerization reactor of claim 1, wherein in (e), a mixing ratio between the polymer dissolved solution and the remaining recovered solvent is 1:1 to 7.

8. The method for removing polymers in an oligomerization reactor of claim 2, wherein in (d), a flow rate ratio between the part of the separated part and the C10 or higher hydrocarbons is 1:0.5 to 3.

9. The method for removing polymers in an oligomerization reactor of claim 1, wherein a flow rate ratio between the heated recovered solvent and the remaining recovered solvent of the separated solvent of (c) is 1:20 to 150.

10. The method for removing polymers in an oligomerization reactor of claim 1, wherein in (e), the polymer dissolved solution and the remaining recovered solvent are mixed in a polymer precipitation device.

11. The method for removing polymers in an oligomerization reactor of claim 1, wherein in (f), the solvent separated in the polymer separator is recovered and reused in the oligomerization reaction of (a).

12. The method for removing polymers in an oligomerization reactor of claim 1, further comprising: after (b), a monomer recovery step of separating unreacted monomers included in the oligomer from which the polymers have been removed using a gas-liquid separator and recovering the monomers to the reactor.

13. The method for removing polymers in an oligomerization reactor of claim 1, wherein the solvent includes an aliphatic hydrocarbon or an aromatic hydrocarbon.

14. The method for removing polymers in an oligomerization reactor of claim 1, wherein in (a), a plurality of reactors is included.

15. The method for removing polymers in an oligomerization reactor of claim 14, wherein the plurality of reactors is arranged in parallel.

16. The method for removing polymers in an oligomerization reactor of claim 1, further comprising: after (d), transferring the part of the heated recovered solvent to the polymer removal device and dissolving a residual polymer in the polymer removal device to prepare the polymer dissolved solution.

17. The method for removing polymers in an oligomerization reactor of claim 1, further comprising: after (a), injecting a catalyst deactivator into the oligomer product solution.

18. The method for removing polymers in an oligomerization reactor of claim 17, wherein the catalyst deactivator includes water, an alcohol-based compound, or a combination thereof.
